SEQUENCE LISTING (3 sequences)

SEQ ID NO: 1 - protein, 16 aa, artificial sequence
  H4 peptide (acyl-peptide)
SGRGKGGKGLGKGGAK

SEQ ID NO: 2 - protein, 13 aa, artificial sequence
  JMH1 peptide (acyl-peptide)
KGGFGGRGGFGGK

SEQ ID NO: 3 - protein, 19 aa, artificial sequence
  R3 peptide (acyl-peptide)
GGRGGFGGRGGFGGRGGFG

(19) United States
(12) Patent Application Publication
(10) Pub. No.: US 2010/0291605 A1
(43) Pub. Date: Nov. 18, 2010

(54) ASSAYS FOR S-ADENOSYLMETHIONINE-DEPENDENT METHYLTRANSFERASES

(75) Inventors: Zhaohui Zhou, Pullman, WA (US); Kathleen Dorgan, Pullman, WA (US)

Correspondence Address: DAVIS WRIGHT TREMAINE, LLP/Seattle, 1201 Third Avenue, Suite 2200, SEATTLE, WA, 98101-3045, US

(73) Assignee: WASHINGTON STATE UNIVERSITY RESEARCH FOUNDATION, Pullman, WA (US)

(21) Appl. No.: 12/161,795

(22) PCT Filed: Jan. 23, 2007
(86) PCT No.: PCT/US2007/060930

§ 371 Date: Aug. 4, 2010

Related U.S. Application Data
(60) Provisional application No. 60/761,170, filed on Jan. 23, 2006.

(51) Int. Cl. C12Q 1/48; C12Q 1/34

(52) U.S. Cl. 435/15; 435/18

(57) ABSTRACT

Disclosed are novel methyltransferase assay methods, comprising: including, in a reaction mixture for a methyltransferase activity, a purified or recombinant adenosine nucleosidase activity that catalyses release of an adenine or adenine derivative moiety from a transmethylation product, and a purified or recombinant adenine deaminase activity that catalyses deamination of the released moiety to hypoxanthine or respective derivative and ammonia, wherein the methyltransferase activity is rate-limiting; and determining the methyltransferase activity by spectrophotometric or chromatographic monitoring of the coupled deamination reaction products, or of subsequent enzymatic or chemical reactions coupled thereto. Coupled oxidation of the hypoxanthine to uric acid and hydrogen peroxide is optionally affected using purified or recombinant xanthine oxidase, wherein the methyltransferase activity is rate-limiting, and wherein determining the methyltransferase activity comprises monitoring of the coupled oxidation reaction. Variations are disclosed comprises monitoring of reaction products (e.g., to detect NH3, Hypoxanthine, H2O2, and Uric Acid).

### STATEMENT REGARDING FEDERALLY FUNDED RESEARCH

The invention was made with government support under the National Institute of Allergy and Infectious Diseases grant no. 1R01AI058146, and the United States government has certain rights in this invention.

### FIELD OF THE INVENTION

Particular aspects relate generally to novel methods and compositions having substantial utility for evaluating the activity and/or kinetic attributes of S-Adenosylmethionine (AdoMet)-dependent methyltransferases (AdoMet), and in particular embodiments to the novel use of recombinant coupling enzymes in enzyme-coupled assays for AdoMet-dependent methyltransferases.

### BACKGROUND

Methyltransferases. S-Adenosyl-L-methionine (AdoMet/SAM)-dependent methyltransferases play an important role in biological systems, including signal transduction, protein repair, biosynthesis, chromatin regulation, and gene silencing (Schubert et al., Trends Biochem. Sci. 28:329-335, 2003; Cheng, X., R. M. Blumenthal (Eds.), S-adenosylmethionine-dependent methyltransferases: structures and functions, World Scientific Publishing Company, Singapore, 1999). Additionally, small molecule, RNA, DNA, lipid, and protein methyltransferases exist (Cheng, X., R. M. Blumenthal (supra); Cheng and Robert, Nucleic Acids Res. 29:3784-3795, 2001; Fujioka, M., Int. J. Biochem. 24:1917-1924, 1992; and Zhang and Reinberg, Genes Dev. 15:2343-2360, 2001). Moreover, data supporting the idea that protein arginine methylation plays a more dynamic role in the histone code has been put forth (Sarmento et al., J. Cell Sci. 117:4449-4459, 2004; Cuthbert et al., Cell 118:545-553, 2004; Bauer et al., EMBO Rep. 3:39-44, 2002; and Wang et al., Science 306:279-83, 2004). However, defining how the protein methyltransferases work and what determines which proteins/residues will become methylated is pivotal for understanding the roles these enzymes play in biology, and methods to evaluate the methylation rate and relative affinity for various substrates are of prominent importance to the development of this understanding.

Deficiencies of the art. Current art-recognized methyltransferase activity assays are primarily based on radioactive labeling using the AdoMet substrate labeled with ¹⁴C or ³H (Patnaik et al., J. Biol. Chem. 279:53248-53258, 2004; Creveling and Daly, Methods Biochem. Anal. 153-182, 1971; and Frankel et al., J. Biol. Chem. 277:3537-3543, 2002). This is because there is very little detectable spectral change between the AdoMet substrate and its common transmethylation product, S-adenosylhomocysteine (AdoHcy/SAH). By nature, radioactive assays require subsequent separation of the product and substrate, which is expensive and time-consuming. Additionally, a significant problem associated with this technique is that in many cases, the AdoHcy product acts as a potent feedback inhibitor to the methyltransferase, adding to the overall margin of error experienced in determining its kinetic parameters (S. Clarke, K. Banfield, in: R. Carmel, D. W. Jacobsen (Eds.) Homocysteine in Health and Disease, Cambridge University Press, New York, 2001, pp. 63-78; Ames et al., J. Med. Chem. 29:354-358 (1986); Hendricks et al., An enzyme-coupled colorimetric assay for S-adenosylmethionine-dependent methyltransferases, Anal. Biochem. 326:100-105, 2004; and Cannon et al., A stereospecific colorimetric assay for (S,S)-adenosylmethionine quantification based on thiopurine methyltransferase-catalyzed thiol methylation, Anal. Biochem. 308:358-363, 2002).

Additionally, two discontinuous assays make use of recombinant coupling enzymes that hydrolyze AdoHcy to homocysteine, which is detected by either chromogenic (Hendricks et al., Anal. Biochem. 326:100-105, 2004) or fluorescent (Wang et al., Biochem. Biophys. Res. Commun. 331:351-356, 2005) thiol-reactive reagents. However, while these assays demonstrate that AdoHcy nucleosidase effectively cleaves the AdoHcy transmethylation product eliminating certain error associated with product inhibition, they have limited utility because of the presence in many reaction mixtures of other molecules that mask or complicate the absorption spectrum used to monitor the reactions.

There is a pronounced need in the art for novel, more diversified and facile methods for determination of methyltransferase activity. There is a pronounced need in the art for identification of additional suitable purified and/or recombinant coupling enzymes for use in such assays. There is a pronounced need in the art for identification of additional purified and/or recombinant coupling enzymes that have suitable kinetic characteristics for coupling to methyltransferase activity assays. There is a pronounced need in the art for identification of additional suitable purified and/or recombinant coupling enzymes that are easy to express and purify in significant quantities to provide for efficient cost-effective assays and kits.

### SUMMARY OF THE INVENTION

Modification of small molecules and proteins by methyltransferases impacts a wide range of biological processes. Herein disclosed are novel and substantially useful enzyme-coupled continuous spectrophotometric assays to quantitatively characterize methyltransferase activity (e.g., S-adenosyl-L-methionine (AdoMet/SAM)-dependent methyltransferase activity, and other methyltransferase activities that give rise to transmethylation products that comprises an adensosine moiety or a derivative thereof).

Particular aspects provide quantitative methods for assaying methyltransferase activity, comprising: including, in a reaction mixture having a methyl donor substrate and a methyltransferase activity that catalyses conversion of the methyl donor substrate to a transmethylation product that comprises an adensosine or adenosine derivative moiety, a purified or recombinant adenosine nucleosidase activity that catalyses release of the respective adenine or adenine derivative moiety from the transmethylation product, and a purified or recombinant adenine deaminase activity that catalyses deamination of the released moiety to hypoxanthine or respective derivative thereof and ammonia, wherein the methyltransferase activity is rate-limiting with respect to the coupled nucleosidase and deamination reactions; and determining the methyltransferase activity by spectrophotometric or chromatographic monitoring of the coupled deamination reaction products, or of subsequent enzymatic or chemical reactions coupled thereto.

In further aspects, the assay methods further comprise coupled oxidation of the hypoxanthine to uric acid and hydrogen peroxide using purified or recombinant xanthine oxidase, wherein the methyltransferase activity is rate-limiting with respect to the coupled nucleosidase, deamination and oxidation reactions, and wherein determining the methyltransferase activity comprises spectrophotometric or chromatographic monitoring of the coupled oxidation reaction.

Particular exemplary embodiments provide enzyme-coupled continuous spectrophotometric assays to quantitatively characterize S-adenosyl-L-methionine (AdoMet/SAM)-dependent methyltransferase activity. In such exemplary embodiments, S-adenosyl-L-homocysteine (AdoHcy/SAH), the transmethylation product of AdoMet-dependent methyltransferases, is hydrolyzed to S-ribosylhomocysteine and adenine by a recombinant S-adenosylhomocysteine/5′-methylthioadenosine nucleosidase activity (e.g., SAHN/MTAN, EC 3.2.2.9). Subsequently, according to preferred embodiments, adenine generated from AdoHcy is further hydrolyzed to hypoxanthine and ammonia by a recombinant adenine deaminase activity (e.g., EC 3.5.4.2). This deamination is associated with a decrease in absorbance at 265 nm that can be monitored continuously. The disclosed coupling enzymes are recombinant (e.g., fusion-tagged recombinants) and easily purified.

In particular embodiments, the adenosine nucleosidase activity is rate-limiting with respect to the coupled deamination and oxidation reactions. In certain embodiments, the purified or recombinant xanthine oxidase is included in the reaction mixture.

In particular embodiments, the adenine deaminase and/or the xanthine oxidase activity comprises or is a recombinant activity, or a recombinant fusion-tagged adenine deaminase or xanthine oxidase activity.

In particular embodiments, the methods further comprise conversion of the hydrogen peroxide to water and oxygen using purified or recombinant catalase.

In additional embodiments, the methods further comprise peroxidation of the hydrogen peroxide using purified or recombinant peroxidase, wherein determining the methyltransferase activity comprises spectrophotometric or chromatographic monitoring of the peroxidation reaction.

Additional embodiments provide a kit for assaying of methyltransferase activity; comprising: a purified or recombinant adenosine nucleosidase activity suitable to catalyse release of an adenine or adenine derivative moiety from a transmethylation product of a transmethylation reaction; and a purified or recombinant adenine deaminase activity suitable to catalyse deamination of the released moiety to hypoxanthine or respective derivative thereof and ammonia, wherein the methyltransferase activity is rate-limiting with respect to the nucleosidase and deamination activities. In particular aspects, the adenosine nucleosidase activity is rate-limiting with respect to the deamination activity.

In additional embodiments the kits further comprise a purified or recombinant xanthine oxidase suitable to oxidize hypoxanthine to uric acid and hydrogen peroxide, wherein the methyltransferase activity is rate-limiting with respect to the nucleosidase, deamination and oxidation activities.

In yet further embodiments the kits further comprise a purified or recombinant peroxidase to catalyze peroxidation of the hydrogen peroxide, wherein determining the methyltransferase activity comprises spectrophotometric or chromatographic monitoring of the peroxidation reaction.

In particular aspects, the adenosine nucleosidase activity is rate-limiting with respect to the deamination, oxidation and peroxidation activities.

### BRIEF DESCRIPTION OF THE DRAWINGS

FIG. 1 shows, according to particular exemplary aspects of the present invention, the absorbance change associated with the conversion of AdoHcy to uric acid. The up arrow indicates the increase in absorbance at 295 nm with time while the down arrow indicates the decrease in absorbance at 260 nm with time.

FIG. 2 shows, according to particular exemplary aspects of the present invention, the time-dependent conversion of AdoHcy to uric acid. AdoHcy (55 μM) pre-incubated with adenine deaminase, MnSO₄, and xanthine oxidase for 4.5 minutes before the reaction was initiated with AdoHcy nucleosidase. The reaction was monitored continuously on a UV-visible spectrophotometer at 295 nm.

FIGS. 3A and 3B show, according to particular exemplary aspects of the present invention, the absorbance change associated with the conversion of AdoHcy to uric acid in the presence of 1 mM dNTP. AdoHcy (50.2 μM) was pre-incubated with dNTP (1 mM), adenine deaminase, MnSO₄, and xanthine oxidase for 2.0 minutes before the reaction was initiated with AdoHcy nucleosidase. The reaction was monitored continuously on a UV-visible spectrophotometer at 297 nm. The up arrow indicates the increase in absorbance at 297 nm with. FIG. 3B is an expanded view of FIG. 3A.

FIG. 4 shows, according to particular exemplary aspects of the present invention, the time-dependent conversion of AdoHcy to uric acid in the presence of 1 mM dNTP. AdoHcy (50.2 μM) was pre-incubated with dNTP (1 mM), adenine deaminase, MnSO₄, and xanthine oxidase for 2.0 minutes before the reaction was initiated with AdoHcy nucleosidase. The reaction was monitored continuously on a UV-visible spectrophotometer at 297 nm.

FIG. 5 shows, according to particular exemplary aspects of the present invention, the change in absorbance of AdoHcy to uric acid in the presence of dNTP (1 mM each of dATP, dTTP, dGTP and dCTP). Varying concentrations of AdoHcy were pre-incubated with dTNP, adenine deaminase, MnSO₄, and xanthine oxidase for 2.0 minutes before the reactions were initiated with AdoHcy nucleosidase. Reactions were monitored continuously on a UV-visible spectrophotometer at 297 nm.

FIG. 6 shows, according to particular exemplary aspects of the present invention, the absorbance change associated with the conversion of ABTS to ABTS radical in the presence of H₂O₂ from the conversion of hypoxanthine to uric acid. The up arrows indicate an increase in absorbance with time while the down arrow indicates a decrease in absorbance with time.

FIGS. 7A and 7B show, according to particular exemplary aspects of the present invention, the absorbance change associated with the conversion of adenine to hypoxanthine catalyzed by adenine deaminase over 5 minutes (7A). The reaction mixture contained 54.3 μM adenine, 1050 μM MnSO₄, and 0.02 μM adenine deaminase buffered in 200 mM Tris pH 8.0 and 37° C. The reaction was completed in 5 min. The arrow indicates the decrease in absorbance at 265 nm with time. In FIG. 7B, the spectrum of the original solution of adenine (dotted line), the reaction at completion (bold solid line) and difference spectrum (thin solid line) are shown.

FIG. 8 shows, according to particular exemplary aspects of the present invention, the linear correlation between the absorbance change at 265 nm and AdoHcy concentration. The assay monitoring the conversion of AdoHcy to hypoxanthine was performed using varying amounts of AdoHcy, 110 μM MnSO₄, 28 nM adenine deaminase, and 17.3 nM AdoHcy nucleosidase in 50 mM potassium phosphate pH 7.4.

FIG. 9 shows, according to particular exemplary aspects of the present invention, that the PRMT1 activity with R3 peptide is dependent upon PRMT1 concentration. Reaction mixtures contained 59 μM AdoMet, 1050 μM MnSO₄, 0.02 μM adenine deaminase, 168 μM AdoHcy nucleosidase, and 211 μM R3 peptide in 50 mM potassium phosphate pH 7.0. Reactions also contained 0 (closed circles) 3.9 (open circles), 7.7 (closed squares), 11.4 (open squares), and 14.9 (open triangles) μM PRMT1, respectively. The inset shows that activity (ΔAbs 265 nm/min) is a function of protein concentration.

FIG. 10 shows, according to particular exemplary aspects of the present invention, an HPLC chromatogram confirming reaction products. The top trace shows an HPLC chromatogram of a mixture of authentic 80 μM samples of AdoMet, AdoHcy, adenine (Ade), and hypoxanthine (Hxan). The middle trace is an HPLC chromatogram of an aliquot of the reaction mixture prior to the addition of R3 peptide. The bottom trace is the chromatogram of an aliquot of the reaction mixture when there was a change at 265 nm. An impurity from AdoMet (I) is seen in the bottom two traces. All traces were monitored at 245 nm.

FIG. 11 shows, according to particular exemplary aspects of the present invention, that the continuous assay monitors methylation of histone 4 protein. Reactions containing 250 μM AdoMet, 10 μM MnSO4, 10 nM AdoHcy nucleosidase, 0.02 μM adenine deaminase, and 0 (open circles) or 5.0 μM (closed circles) of purified [30] histone 4 protein were equilibrated to 37° C. for 10 minutes and initiated with 4 μM PRMT1. The decrease in absorbance associated with the methylation of histone 4 protein was monitored at 265 nm.

FIG. 12A shows, according to particular exemplary aspects of the present invention, reaction of PIMT with AdoMet and B-DSIP (see Example 4). Spectra were collected over 6 hours and the reaction was monitored at 265 nm.

FIG. 12B shows, according to particular exemplary aspects of the present invention, reaction of PIMT with AdoMet and KASA-isoD-LAKY peptide (see Example 4). Spectra were collected over 6 hours and the reaction was monitored at 265 nm.

### DETAILED DESCRIPTION

Particular exemplary aspects provide novel enzyme-coupled assays for methyltransferases (e.g., including AdoMet-dependent methyltransferases, and other methyltransferase activities that give rise to transmethylation products that comprises an adensosine moiety or a derivative thereof). For example, the inventive methods can be used to assay other enzymes that produce AdoHcy, 5′-methylthioadenosine, or compounds that can be cleaved by AdoHcy nucleosidase. The understanding of certain exemplary inventive aspects is facilitated with reference to reaction schemes 1-3 below, which are referred to herein.

The following DEFINITIONS are also provided:

AdoHcy/SAH refers to S-adenosyl-L-homocysteine;

AdoMet/SAM refers to S-adenosyl-L-methionine;

Hepes refers to N-(2-hydroxyethyl)piperazine-N′-2-ethanesulfonic acid;

HPLC refers to high-performance liquid chromatograph;

H4 peptide refers to _{acyl}SGRGKGGKGLGKGGAK (SEQ ID NO:1);

IPTG refers to isopropyl-β-_{D}-thiogalactopyranoside;

JMH1 peptide refers to _{acyl}KGGFGGRGGFGGK (SEQ ID NO:2);

LB refers to Luria-Bertani;

MTA refers to 5′-methylthioadenosine;

PRMT1 refers to protein arginine N-methyltransferase 1;

R3 peptide refers to _{acyl}GGRGGFGGRGGFGGRGGFG (SEQ ID NO:3); and

Tris refers to tris(hydroxymethyl)aminomethane.

“Methyl donor substrate” as used herein refers to a transmethylation substrate (methyl donor) for a methyltransferease enzymatic activity, including but not limited to the methyl donor substrate S-adenosyl-L-methionine (AdoMet/SAM).

“Methyltransferase activity” as used herein refers to a methyltransferease enzymatic activity that catalyzes transfer of a methyl group from a methyl donor substrate (e.g., from S-adenosyl-L-methionine (AdoMet/SAM)) to a methyl group recipient molecule (e.g., peptide, protein nucleic acid, etc.) and converting the methyl donor substrate into a a transmethylation product. Such methyltransferase activities include, but are not limited to S-adenosyl-L-methionine (AdoMet/SAM)-dependent methyltransferase activities.

“Transmethylation product” as used herein refers to the conversion product of the methyltransferase activity on the methyl donar substrate, includes, but is not limited to S-adenosylhomocysteine (AdoHcy), 5′-methylthioadenosine (MTA), and structural analogs of AdoHcy or MTA with hydrophobic residues at the C5 position.

“Adensosine moiety or derivative thereof” as used herein refers to adenosine or adenosine derivates that are suitable substrates for an adenosine nucleosidase activity.

“Adenosine nucleosidase activity” or “recombinant adenosine nucleosidase activity” as used herein refers to a purified or recombinant nucleosidease enzymatic activity that is capable of releasing adenine or a derivative thereof from at substrate adensosine or adenosine derivative moiety.

“Adenine deaminase activity” or “recombinant adenine deaminase activity” as used herein refers to a purified or recombinant deaminase activity that is capable of releasing ammonia from adenine or from an adenine derivative, and includes but is not limited to adenine deaminase activity that converts adenine to hypoxanthine and ammonia.

“Xanthine oxidase” or “recombinant xanthine oxidase” as used herein refers to a purified or recombinant oxidase enzymatic activity capable of converting hypoxanthine or a derivative thereof to uric acid (or respective derivative) and hydrogen peroxide, and includes but is not limited to xanthine oxidase activity that converts hypoxanthine to uric acid and hydrogen peroxide.

“Peroxidase” or ‘recombinant peroxidase’ as used herein refers to a purified or recombinant peroxidase enzymatic activity capable of catalyzing the oxidation of organic substrates in the presence of a peroxide, and includes but is not limited to hydrogen peroxidase that catalyses the oxidation of organic substrates in the presence of hydrogen peroxide.

“Spectrophotometric monitoring” as used herein refers to spectroscopic methodology (e.g., UV-vis, Fluorescence, Vibrational, Mass) used to monitor the concentration of a chemical species present within the reaction mixture either through direct analysis of the reaction mixture or analysis of a quenched aliquot.

“Chromatographic monitoring” as used herein refers to the use of chromatographic methodology (e.g., High Performance Liquid Chromatography, Liquid Chromatography, Gas Chromatography) used to monitor the concentration of a chemical species present within the reaction mixture either through direct analysis of the reaction mixture or analysis of a quenched aliquot.

“Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) as described herein additionally includes functional variants (including conservative amino acid sequence variants as described herein), fragments, muteins, derivatives and fusion proteins thereof. It will be appreciated that the methods of the present inventions are not limited to the use of any particular enzymatic activity in these categories, but can be practiced with any suitable enzymatic activity having the requisite functional relied upon.

TABLE 1
Summary of Exemplary EC and accession numbers:

| MOLECULE | PROTEIN |
|---|---|
| EC No. | ACCESSION NO.: |
| S- | CAC13509 |
| adenosylhomocysteine | G84954 |
| (AdoHcy) | AAK05978 |
| nucleosidase activity | CAM08202 |
| comprises AdoHcy | CAA98927 |
| nucleosidase EC | NP_853762 |
| 3.2.2.9 | NP_214605 |
| | CAD92956 |
| | AAL19171 |
| | YP_765922 |
| | CAK05806 |
| | Etc. |
| adenine deaminase | BAC51407 |
| activity comprises | BAC48430 |
| adenine deaminase EC | YP_767615 |
| 3.5.4.2 | CAK07507 |
| | YP_770041 |
| | YP_769461 |
| | YP_769121 |
| | CAK09957 |
| | CAK09373 |
| | CAK09029 |
| | NP_772782 |
| | NP_769805 |
| | NP_562184 |
| | BAB80974 |
| | Etc. |
| xanthine oxidase | S66603 |
| comprises xanthine | |
| oxidase EC 1.1.3.22 | |
| peroxidase EC | CAA40796 |
| 1.11.1.1-15 | BAF33313 |
| | BAF33314 |
| | BAF33315 |
| | BAF33316 |
| | BAF33317 |
| | P00434 |
| | CAH67141 |
| | CAJ86371 |
| | CAJ86421 |
| | ABC02343 |
| | ABK28706 |
| | ABK59095 |
| | BAA14143 |
| | AAA72223 |
| | 1ATJ_A |
| | CAA00083 |
| | Etc. |
| Catalase EC 1.11.1.6 | CSRZ |
| | A40662 |
| | A47685 |
| | AB0708 |
| | AD3621 |
| | JC7672 |
| | T45091 |
| | T42369 |
| | JE0126 |
| | S65793 |
| | A55092 |
| | S71112 |
| | A49388 |
| | S40265 |
| | CAB58320 |
| | CAB61183 |
| | AAO51894 |
| | AAA40884 |
| | AAA33441 |
| | Etc. |

### Biologically Active Variants

Variants of “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) as described herein have substantial utility in various aspects of the present invention. Variants can be naturally or non-naturally occurring. Naturally occurring variants are found in humans or other species and comprise amino acid sequences which are substantially identical to the amino acid sequences exemplified by EC 3.2.2.9, EC 3.5.4.2, EC 1.1.3.22 and EC 1.11.1.1-15, and include natural sequence polymorphisms. Species homologs of the protein can be obtained using subgenomic polynucleotides to make suitable probes or primers for screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants, are also included here. Preferably, naturally or non-naturally occurring variants have amino acid sequences which are at least 85%, 90%, or 95% identical to the amino acid sequences exemplified by EC 3.2.2.9, EC 3.5.4.2, EC 1.1.3.22 and EC 1.11.1.1-15. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 1:

TABLE 2
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in Table 2 of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In particular aspects, functional equivalents of the enzymatic activities of the present inventive methods can have from 1, to about 3, to about 5, to about 10, or to about 20 conservative amino acid substitutions, and retain suitable activity.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

Such substitutions may be made in accordance with those set forth in TABLE 3 as follows:

TABLE 3

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

“Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) as described herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (Mark et al., U.S. Pat. No. 4,959,314).

Preferably, amino acid changes in the “Adenosine nucleosidase activity,” “Adenine deaminase activity,” “Xanthine oxidase,” and “Peroxidase” variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. In certain aspects, properties and functions of “Adenosine nucleosidase activity,” “Adenine deaminase activity,” “Xanthine oxidase,” and “Peroxidase” variants are of the same type as a protein comprising the amino acid sequence encoded by the nucleotide sequences exemplified by EC 3.2.2.9, EC 3.5.4.2, EC 1.1.3.22 and EC 1.11.1.1-15, although the properties and functions of variants can differ in degree.

“Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) variants include glycosylated forms, aggregative conjugates with other molecules, and covalent conjugates with unrelated chemical moieties (e.g., pegylated molecules). “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) variants also include allelic variants (e.g., polymorphisms), species variants, and muteins. Truncations or deletions of regions which do not preclude functional activity of the proteins are also variants. Covalent variants can be prepared by linking functionalities to groups which are found in the amino acid chain or at the N- or C-terminal residue, as is known in the art.

It will be recognized in the art that some amino acid sequence of the “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptides of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. Thus, the “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptides of the present invention may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic (Pinckard et al., Clin. Exp. Immunol. 2:331-340, 1967; Robbins et al., Diabetes 36:838-845, 1987; Cleland et al., Crit. Rev. Therapeutic Drug Carrier Systems 10:307-377, 1993).

Amino acids in “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptides of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904, 1992 and de Vos et al. Science 255:306-312, 1992).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptide will not be more than 50, 40, 30, 25, 20, 15, 10, 5 or 3.

In addition, pegylation of “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptides and/or muteins is expected to provide such improved properties as increased half-life, solubility, and protease resistance. Pegylation is well known in the art.

### Fusion Proteins

Fusion proteins comprising proteins or polypeptide fragments of “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptides can also be constructed. Fusion proteins are useful for generating antibodies against amino acid sequences and for use in various purification targeting and assay systems. Physical methods, such as protein affinity chromatography, or library-based assays for protein-protein interactions, such as the yeast two-hybrid or phage display systems, can also be used for this purpose. Such methods are well known in the art and can also be used as in purification schemes. Fusion proteins comprising a signal sequences can be used.

A fusion protein comprises two protein segments fused together by means of a peptide bond. Amino acid sequences for use in fusion proteins of the invention can be utilize the amino acid sequences exemplified by EC 3.2.2.9, EC 3.5.4.2, EC 1.1.3.22 and EC 1.11.1.1-15 or can be prepared from biologically active variants of exemplified EC 3.2.2.9, EC 3.5.4.2, EC 1.1.3.22 and EC 1.11.1.1-15, such as those described above. The first protein segment can include of a full-length “Adenosine nucleosidase activity” (EC 3.2.2.9), “Adenine deaminase activity” (EC 3.5.4.2), “Xanthine oxidase” (EC 1.1.3.22), and “Peroxidase” (EC 1.11.1.1-15) polypeptide.

Other first protein segments can consist of a portion of the contiguous amino acids from exemplified by EC 3.2.2.9, EC 3.5.4.2, EC 1.1.3.22 and EC 1.11.1.1-15.

The second protein segment can be a full-length protein or a polypeptide fragment. Proteins commonly used in fusion protein construction include β-galactosidase, glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequences of exemplified by EC 3.2.2.9, EC 3.5.4.2, EC 1.1.3.22 and EC 1.11.1.1-15 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

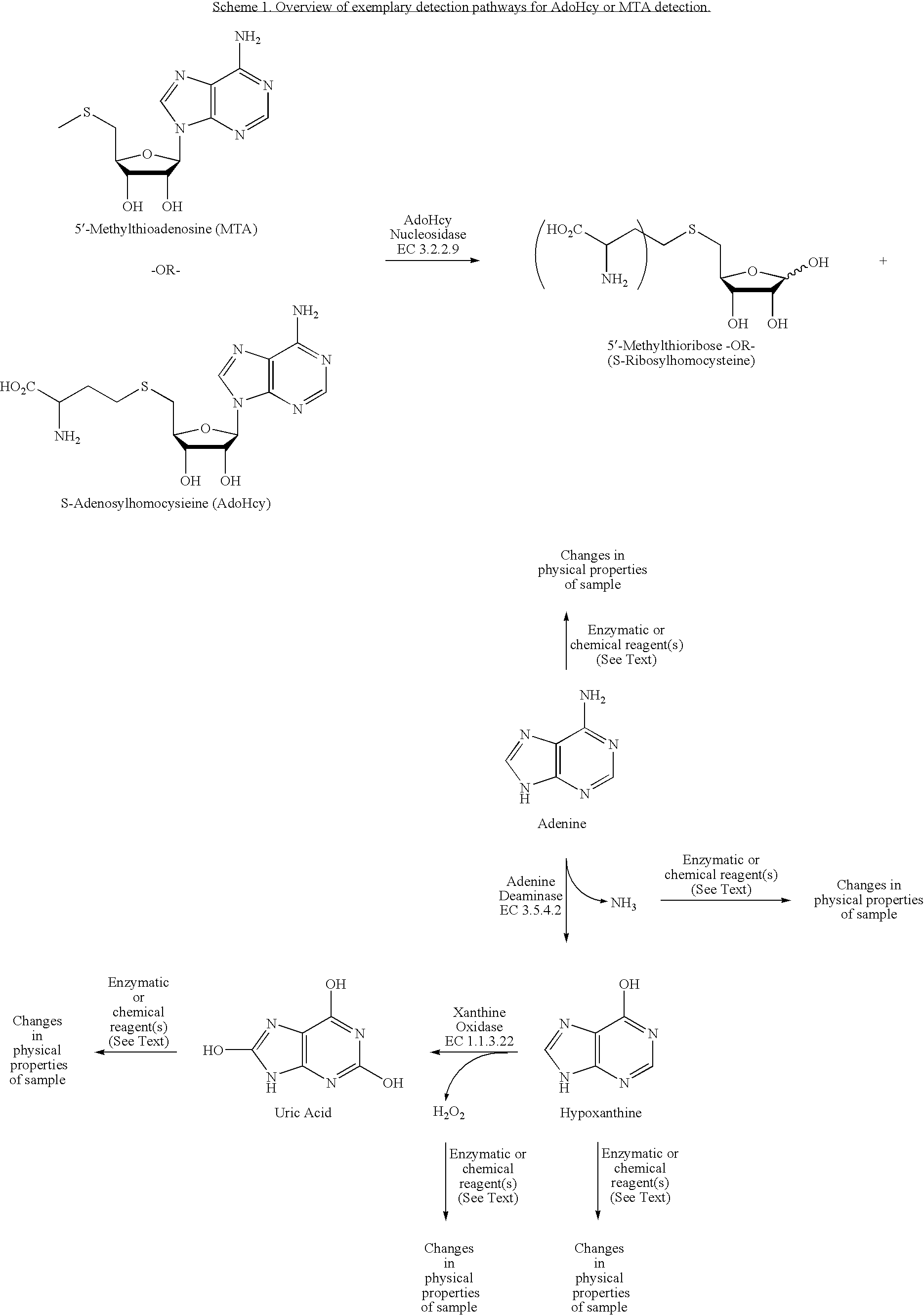

The core of the enzyme-coupled assays comprise cleavage of the transmethylation products using a purified or recombinant adenosine nucleosidase activity that catalyses release of an adenine moiety or derivative thereof from the transmethylation product. For example, AdoHcy Nucleosidase (EC 3.2.2.9)), which catalyses the release of adenine from 5-methylthioadenosine (MTA) or AdoHcy (Scheme 1), can be used.

According to particular inventive aspects, once adenine is released and the feedback inhibition of the methyltransferase is eliminated many chemical and enzymatic detection schemes become viable to those skilled in the art. For example, detection can derive from reaction(s) of adenine and/or the chemical species deriving thereof utilizing enzymatic turnover, redox reactions, metal complexation, and/or interactions/reactions with chemical agents that result in a detectable change in the physical or spectroscopic properties of the reagent mixture.

The potential for modulation of the detection scheme provides additional aspects of the invention. Particular exemplary embodiments provided herein relate to a “continuous” spectrophotometric monitoring, wherein an intensity of absorbance at a particular wavelength is recorded as a function of time. From these data, concentrations of a particular reagent or product can be determined. Similarly, other spectrophotometric methods (e.g., Fluorescence, Vibrational, and Mass) can provide the same quantitative information. The specific type of spectrophotometric method and spectral frequencies utilized in quantization will be dictated by both the medium and the particular reagent or product.

Additional embodiments provide methods for “non-continuous” spectrophotometric or chromatographic monitoring. In non-continuous detection schemes, aliquots of the reagent mixture are removed and quenched at specific time intervals. Quenching results in the cessation of enzymatic activity (e.g., through the addition of agents to denature the enzymes or block the activity of one or more of the enzymes). Once an aliquot has been quenched, spectrophotometric or chromatographic methods that may or may not involve further chemical treatment are utilized to quantitate the chemical products of interest. The specific type of spectrophotometric or chromatographic method utilized in quantitation will be dictated by both the medium and the particular reagent or product.

It is to be understood that, given the flexible nature of this enzymatic assay and the wide range of AdoMet-dependent methyltransferase enzymes, variations from the core methodology as described herein adopted to tailor the detection scheme to specific AdoMet-dependent methyltransferases enzymes and/or media containing such enzymes are anticipated by this disclosure. The spectroscopic, physical and chemical characteristics of the reagents, products and media will dictate the specific variations requires for adaptation to differing AdoMet-dependent methyltransferases enzymes and/or media. The method as disclosed herein provides a substantial number of potential targets from which specific, tailored assays can be constructed. For example, assays tailored to specific enzymes of particular biological importance (e.g., DNA methyltransferases). The method may also be tailored to specific media, for example whole blood and/or plasma to assay pharmacologically important enzymes (e.g., thiopurine s-methyltransferase). Such variants may involve modulation of reagent concentrations, addition of secondary enzymatic or chemical steps to induce physical changes within the sample that can subsequently be quantitated through spectrophotometric or chromatographic methods in either a continuous or non-continuous detection scheme. Immuniological reagents may also be introduced to adapt the method to high throughput enzyme linked immunosorbent assays (ELISA).

In particular aspects (see schemes 1 above and 2 below), a purified or recombinant adenine deaminase activity is included in a coupled fashion, wherein the deaminase catalyses deamination of the released adenine moiety or derivative thereof to produce hypoxanthine or the respective derivative thereof and ammonia, wherein the methyltransferase activity is rate-limiting with respect to the coupled nucleosidase and deamination reactions. In such exemplary embodiments, the methyltransferase activity is monitored and/or determined by spectrophotometric monitoring of the coupled deamination reaction.

As shown in scheme 2 below, AdoHcy can be converted to S-ribosylhomocysteine and adenine by AdoHcy nucleosidase. Earlier studies demonstrated that AdoHcy nucleosidase effectively cleaves the AdoHcy transmethylation product (Hendricks et al., Anal. Biochem. 326:100-105, 2004; and Cannon et al., Anal. Biochem. 308:358-363, 2002), eliminating particular error associated with product inhibition. As disclosed herein, the adenine product of the reaction can then converted to hypoxanthine by the additional coupled enzyme activity adenine deaminase, resulting, for example, in an absorbance decrease at 265 nm that can be easily detected by UV spectrometry. The rapid and continuous detection of the conversion of substrate to product also helps to improve the accuracy over discontinuous assaying.

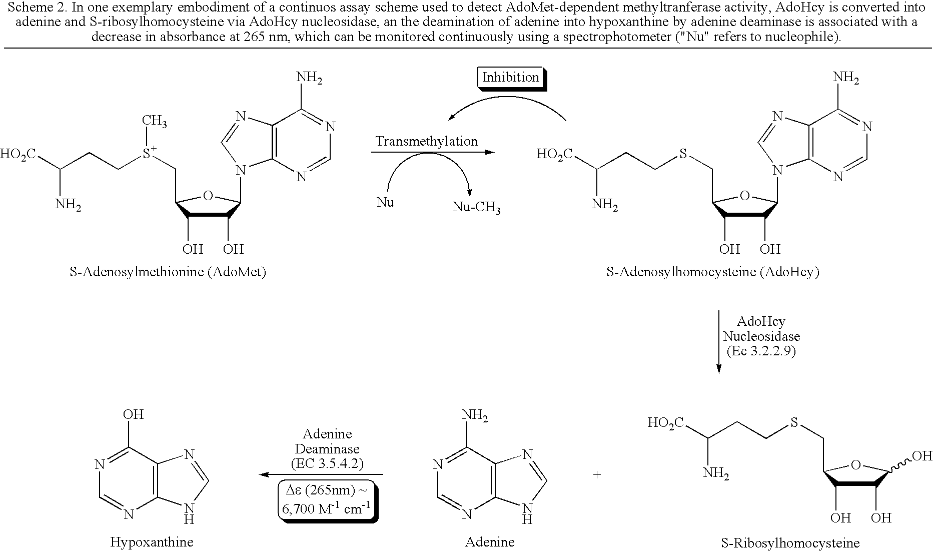

In yet further exemplary embodiments (see schemes 1 above, and 3 below), additional coupling enzymes, such as xanthine oxidase and hydrogen peroxide peroxidase are used, the overall reactions can be monitored, for example at 295 nm or even higher wavelength around 600 nm, which is in the visible region.

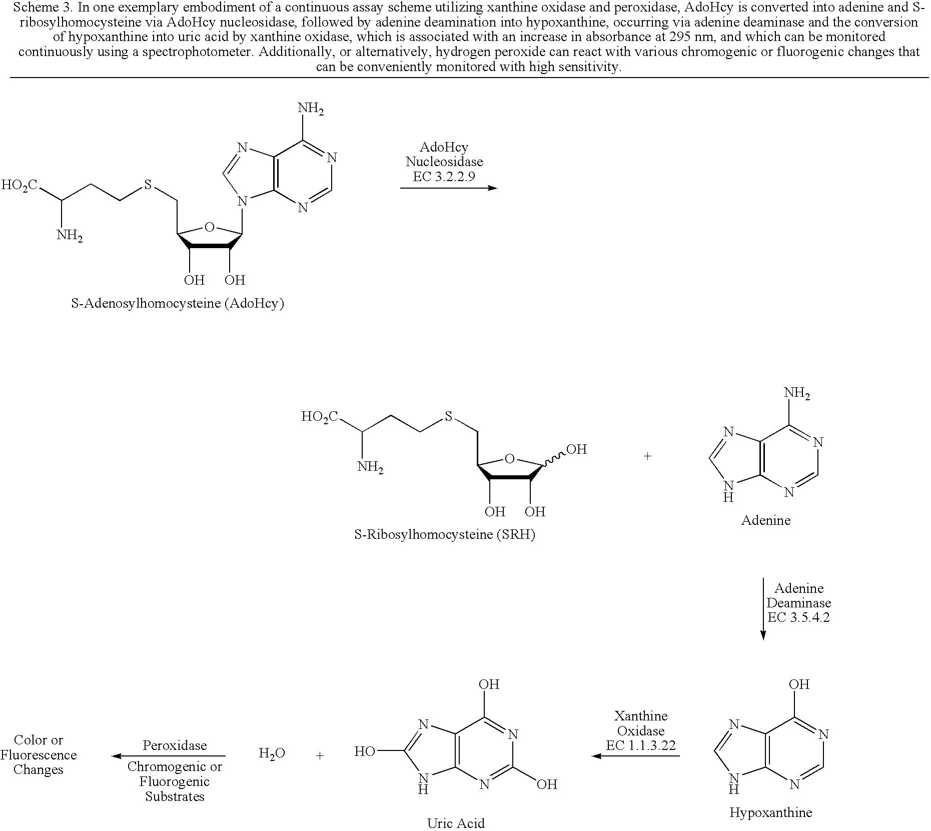

In such exemplary embodiments, therefore, the coupling enzyme, xanthine oxidase, catalyzes the conversion of hypoxanthine to uric acid with a concomitant increase in absorbance at 295 nm, and also produces hydrogen peroxide as an alternative product that can be monitored.

Therefore, in yet further aspects, the hydrogen peroxide is quantified by reactions with various chromogenic or fluorogenic substrates catalyzed by hydrogen peroxide peroxidase. The color or fluorescence changes can be conveniently monitored with high sensitivity. These two additional coupling enzymes have been described in the literature, and the enzymes are commercially available. The inventive assays, therefore, are easily optimized and adapted for a broad range of applications monitored by various spectroscopic methods, including ultraviolet-visible spectrometry and fluorometry (Scheme 3).

Therefore, according to particular exemplary aspects, enzymatic reactions utilizing Adenine Deaminase (EC 3.5.4.2) and Xanthine Oxidase (EC 1.1.3.22) can generate chemical species from which alternative detection schemes can be built (schemes 1 and 3). In such cases, either enzymatic and/or chemical reaction schemes can be implemented for detection.

According to particular aspects, many variations can be used for the detection of any one the products (e.g., NH₃, Hypoxanthine, H₂O₂, and Uric Acid) and based on the teachings disclosed herein, one of ordinary skill in the art is enabled to implement such detection schemes and TABLES 1-3 below proved an exemplary set of detection schemes for use on adenine or the aforementioned derivative products). For example, the TABLES 1-3 provide a partial listing of examples of enzymes and chemical agents that can act on Adenine, NH₃, Hypoxanthine, H₂O₂, and Uric Acid. The specific examples included herein in TABLES 4-6, are given by way of illustration only to demonstrate and teach that many variations on the specific exemplary detection schemes are available to one skilled in the art given the teachings of the instant specification.

TABLE 4
Exemplary enzymatic reactions involving adenine.

| EC | | |
|---|---|---|
| Number | Official Name | Reaction Catalysed |
| 3.5.4.2 | Adenine deaminase. | Adenine + H(2)O <=> hypoxanthine + NH(3) |
| 1.5.99.12 | Cytokinin dehydrogenase | N(6)-dimethylallyladenine + acceptor + H(2)O <=> adenine + |
| | | 3-methylbut-2-enal + reduced acceptor |
| 2.4.2.28 | S-methyl-5-thioadenosine | S-methyl-5-thioadenosine + phosphate <=> adenine + S- |
| | phosphorylase | methyl-5-thio-alpha-D-ribose 1-phosphate |
| 2.4.2.7 | Adenine | AMP + diphosphate <=> adenine + 5-phospho-alpha-D-ribose |
| | phosphoribosyltransferase | 1-diphosphate |
| 2.7.8.25 | Triphosphoribosyl- | ATP + 3-dephospho-CoA <=> 2′-(5″-triphosphoribosyl)-3′- |
| | dephospho-CoA synthase | dephospho-CoA + adenine |
| 3.2.2.4 | AMP nucleosidase | AMP + H(2)O <=> D-ribose 5-phosphate + adenine |
| 3.2.2.7 | Adenosine nucleosidase | Adenosine + H(2)O <=> D-ribose + adenine |
| 3.2.2.9 | Adenosylhomocysteine | 1) S-adenosyl-L-homocysteine + H(2)O <=> S-(5-deoxy-D- |
| | nucleosidase | ribos-5-yl)-L-homocysteine + adenine |
| | | 2) S-methyl-5′-thioadenosine + H(2)O <=> 5-methyl-5-thio- |
| | | D-ribose + adenine |
| 3.2.2.13 | 1-methyladenosine | 1-methyladenosine + H(2)O <=> 1-methyladenine + D- |
| | nucleosidase. | ribose |
| 3.2.2.16 | Methylthioadenosine | S-methyl-5′-thioadenosine + H(2)O <=> S-methyl-5-thio-D- |
| | nucleosidase | ribose + adenine |
| 3.2.2.20 | DNA-3-methyladenine | Hydrolysis of alkylated DNA, releasing 3-methyladenine |
| | glycosylase I | |
| 3.2.2.21 | DNA-3-methyladenine | Hydrolysis of alkylated DNA, releasing 3-methyladenine, |
| | glycosylase II. | 3-methylguanine, 7-methylguanine and 7-methyladenine |

TABLE 5
Enzymatic reactions involving S-adenosyl-homocysteine (AdoHcy) or
methylthioadenosine (MTA).

| EC | | |
|---|---|---|
| Number | Official Name | Reaction Catalysed |
| 1.16.1.8 | [Methionine synthase] | 2 [methionine synthase]-methylcob(I)alamin + 2 S- |
| | reductase | adenosylhomocysteine + NADP(+) <=> 2 [methionine |
| | | synthase]-cob(II)alamin + NADPH + 2 S-adenosyl-L- |
| | | methionine |
| 2.1.1.1- | S-Adenosylmethionine | S-adenosyl-L-methionine + substrate <=> S-adenosyl-L- |
| 157 | dependent methyltransferases | homocysteine + methylated substrate |
| | (2.1.1.1-157 minus 22 | |
| | entries) | |
| 2.3.1.161 | Lovastatin nonaketide | Acetyl-CoA + 8 malonyl-CoA + 11 NADPH + S-adenosyl-L- |
| | synthase | methionine <=> dihydromonacolin L + 9 CoA + 8 CO(2) + 11 |
| | | NADP(+) + S-adenosyl-L-homocysteine + 6 H(2)O |
| 2.5.1.16 | Spermidine synthase | S-adenosylmethioninamine + putrescine <=> 5′- |
| | | methylthioadenosine + spermidine |
| 2.5.1.22 | Spermine synthase | S-adenosylmethioninamine + spermidine <=> 5′- |
| | | methylthioadenosine + spermine |
| 2.5.1.23 | Sym-norspermidine synthase | S-adenosylmethioninamine + propane-1,3-diamine <=> 5′- |
| | | methylthioadenosine + bis(3-aminopropyl)amine |
| 2.5.1.38 | Isonocardicin synthase | S-adenosyl-L-methionine + nocardicin E <=> 5′- |
| | | methylthioadenosine + isonocardicin A |
| 2.5.1.4 | Adenosylmethionine | S-adenosyl-L-methionine <=> 5′-methylthioadenosine + 2- |
| | cyclotransferase | aminobutan-4-olide |
| 2.5.1.24 | Discadenine synthase. | S-adenosyl-L-methionine + N(6)-(delta(2)-isopentenyl)- |
| | | adenine <=> 5′-methylthioadenosine discadenine |
| 3.3.1.1 | Adenosylhomocysteinase | S-adenosyl-L-homocysteine + H(2)O <=> L-homocysteine + |
| | | adenosine |
| 3.3.1.2 | Adenosylmethionine | S-adenosyl-L-methionine + H(2)O <=> L-homoserine + |
| | hydrolase | methylthioadenosine |
| 3.5.4.28 | S-adenosylhomocysteine | S-adenosyl-L-homocysteine + H(2)O <=> S-inosyl-L- |
| | deaminase | homocysteine + NH(3) |
| 4.4.1.14 | 1-aminocyclopropane-1- | S-adenosyl-L-methionine <=> 1-aminocyclopropane-1- |
| | carboxylate synthase | carboxylate + methylthioadenosine |

TABLE 6
Enzymatic reactions involving hypoxanthine, NH₃ and H₂O₂.

| EC | | |
|---|---|---|
| Number | Official Name | Reaction Catalysed |
| 1.17.1.4 | Xanthine dehydrogenase | Xanthine + NAD(+) + H(2)O <=> urate + |
| | | NADH (works on hypoxanthine) |
| 1.17.3.2 | Xanthine oxidase | Xanthine + H(2)O + O(2) <=> urate + |
| | | H(2)O(2) (works on hypoxanthine) |
| 1.4.1.4 | L-Glutamate Dehydrogenase | α-Ketoglutatate + NH₃ + |
| | | NADPH <=> NADP + L-Glutamate + H₂O |
| 1.11.1.1-15 | Various peroxidase | H₂O₂ + substrate to <=> |
| | | various oxidation products |

According to additional aspects, various chemical methods for ammonia quantization can be used to practice the inventive embodiments. For example, it is recognized that low-level ammonia nitrogen may be present in water naturally as a result of the biological decay of plant and animal matter. Higher concentrations may be found in raw sewage and industrial effluents, particularly from petroleum refineries where ammonia is a by-product of the refining process. Additionally, ammonia is a major component of fertilizers. High concentrations in surface waters can indicate contamination from waste treatment facilities, industrial effluents or fertilizer run off. Excessive ammonia concentrations are toxic to aquatic life. Therefore, various ammonia detection methods have been developed, and are known in the art.

The Nessler Method. In the Nessler method, ammonia concentrations are determined by direct Nesslerization. In some waters, calcium and magnesium concentrations can cause cloudiness of the reagent. Adding a few drops of stabilizer solution (Rochelle Salt) will prevent this cloudiness. Results are expressed as ppm (mg/L) NH3-N. Although the reagent itself is stable, its high alkali content attacks the glass ampoule. The resulting precipitate interferes with color comparison below 1 ppm. We recommend, therefore, stocking quantities of CHEMets® ampoules and VACUettes® ampoules that will be used within 5 months. A 2-month supply of Vacu-vials® ampoules is suggested. Refrigeration will nearly double the shelf-life of these products. Preferably, the samples are distilled prior to analysis (see, e.g., ASTM D 1426-93, Ammonia Nitrogen in Water, Test Method A. APHA Standard Methods, 18th ed., p. 4-78, method 4500-NH3 C (1992)).

The Salicylate Method. Free ammonia reacts with hypochlorite to form monochloramine. Monochloramine then reacts with salicylate, in the presence of sodium nitro-ferricyanide, to form 5-aminosalicylate, a green colored complex. The test method measures free ammonia plus monochloramine, and the results are expressed in ppm (mg/Liter) ammonia-nitrogen, NH₃—N. (see, e.g., Krom, Michael D. (1980) Spectrophotometric Determination of Ammonia: A Study of a Modified Berthelot Reduction Using Salicylate and Dichloroisocyanurate, The Analyst, V105, pp. 305-316); and Methods for the Chemical Analysis of Water and Wastes, March 1979, Method 351.2).

The inventive schemes may involve further enzymatic turnover, redox reactions, metal complexation, and/or interactions/reactions with chemical agents that result in a detectable change in the physical or spectroscopic properties of the reagent mixture. Specific manifestations of this assay are provided herein as illustrations of potential detection pathways and are not intended to limit the scope of the invention as various modifications will become apparent to one skilled in the art.

In the working Examples that follow, particular utilities of the inventive assays are demonstrated and exemplified by characterizing the activity of recombinant rat PRMT1, a protein methyltransferase known to methylate a variety of proteins including histone 4, fibrillarin and RNA binding proteins (see for review, e.g., Bedford and Richard, Arginine methylation: an emerging regulator of protein function, Mol. Cell. Biol. 18:263-272, 2005). More specifically, in particular aspects, the utility of this assay was shown using recombinant rat protein arginine N-methyltransferase 1 (PRMT1, EC 2.1.1.125) which catalyzes the mono- and dimethylation of guanidino nitrogens of arginine residues in select proteins. Using this assay, the kinetic parameters of PRMT1 with three synthetic peptides were determined. As described herein above, an advantage of this assay is the destruction of AdoHcy by AdoHcy nucleosidase, which alleviates AdoHcy product feedback inhibition of S-adenosylmethionine-dependent methyltransferases. Additional advantages are provided by the diversity of detection systems using the enzyme-coupled activities as described herein, which enable detection at a variety of absorption values and reaction conditions. Finally, this method may be used to assay other enzymes that produce AdoHcy, 5′-methylthioadenosine, or compounds that can be cleaved by AdoHcy nucleosidase.

### PARTICULAR PREFERRED EMBODIMENTS

Particular embodiments provide a quantitative method for assaying methyltransferase activity, comprising: including, in a reaction mixture having a methyl donor substrate and a methyltransferase activity that catalyses conversion of the methyl donor substrate to a transmethylation product that comprises an adensosine or adenosine derivative moiety, a purified or recombinant adenosine nucleosidase activity that catalyses release of the respective adenine or adenine derivative moiety from the transmethylation product, and a purified or recombinant adenine deaminase activity that catalyses deamination of the released moiety to hypoxanthine or respective derivative thereof and ammonia, wherein the methyltransferase activity is rate-limiting with respect to the coupled nucleosidase and deamination reactions; and determining the methyltransferase activity by spectrophotometric or chromatographic monitoring of the coupled deamination reaction products, or of subsequent enzymatic or chemical reactions coupled thereto. In certain aspects, the adenosine nucleosidase activity is rate-limiting with respect to the coupled deamination reaction.

In specific embodiments, spectrophotometric monitoring comprises spectrophotometric monitoring of the deamination reaction, and in certain aspects, such spectrophotometric monitoring comprises continuous monitoring of absorbance at 265 nanometers, wherein the progress of deamination is accompanied by decreasing absorbance at 265 nanometers.

In certain embodiments, the methyltransferase activity consists of or comprises S-adenosyl-L-methionine (AdoMet/SAM)-dependent methyltransferase activity. In certain embodiments, the purified or recombinant adenosine nucleosidase activity consists of or comprises a purified or recombinant S-adenosylhomocysteine (AdoHcy) nucleosidase activity. In particular embodiments, the purified or recombinant S-adenosylhomocysteine (AdoHcy) nucleosidase activity consists of or comprises AdoHcy nucleosidase EC 3.2.2.9. In particular embodiments, the purified or recombinant adenine deaminase activity consists of or comprises adenine deaminase EC 3.5.4.2.

In particular aspects, the transmethylation product comprises at least one agent selected from the group consisting of S-adenosylhomocysteine (AdoHcy); 5′-methylthioadenosine (MTA); and structural analogs of AdoHcy or MTA with hydrophobic residues at the C5 position.

In further embodiments, the exemplary inventive methods further comprise coupled oxidation of the hypoxanthine to uric acid and hydrogen peroxide using purified or recombinant xanthine oxidase, wherein the methyltransferase activity is rate-limiting with respect to the coupled nucleosidase, deamination and oxidation reactions, and wherein determining the methyltransferase activity comprises spectrophotometric or chromatographic monitoring of the coupled oxidation reaction. In particular embodiments, the adenosine nucleosidase activity is rate-limiting with respect to the coupled deamination and oxidation reactions. In particular embodiments, the purified or recombinant xanthine oxidase is included in the reaction mixture. In certain aspects, the purified or recombinant xanthine oxidase comprises xanthine oxidase EC 1.1.3.22.

In particular aspects, the spectrophotometric monitoring of the coupled oxidation reaction comprises continuous monitoring of absorbance at 295 or 297 nanometers, wherein the progress of oxidation reaction is accompanied by increasing absorbance at 295 or 297 nanometers.

Certain embodiments further comprise peroxidation of the hydrogen peroxide, wherein determining the methyltransferase activity comprises spectrophotometric or chromatographic monitoring of the peroxidation reaction. In particular embodiments, spectrophotometric monitoring of the peroxidation reaction comprises conversion of 2,2′-azino-bis(3-ethylbenzothiazoline-6-sulfonic acid) (ABTS) to the ABTS radical and monitoring of an increase in absorbance at 413 nm or at a higher wavelength characteristic of the formation of the ABTS radical.

In yet further embodiments, the exemplary inventive methods further comprise conversion of the hydrogen peroxide to water and oxygen using purified or recombinant catalase.

In particular embodiments of the methods, the adenine deaminase activity is a recombinant adenine deaminase activity, and in certain aspects, the recombinant adenine deaminase activity is a recombinant fusion-tagged adenine deaminase.

Additional exemplary embodiments provide kits for spectrophotometric assay of methyltransferase activity; comprising: a purified or recombinant adenosine nucleosidase activity suitable to catalyse release of an adenine moiety or derivative thereof from a transmethylation product of a transmethylation reaction; and a purified or recombinant adenine deaminase activity suitable to catalyse deamination of the released adenine moiety or derivative thereof to hypoxanthine or respective derivative thereof and ammonia, wherein the methyltransferase activity is rate-limiting with respect to the nucleosidase and deamination activities. In particular embodiments, the adenosine nucleosidase activity is rate-limiting with respect to the deamination activity.

Additional kit embodiments further comprise a purified or recombinant xanthine oxidase suitable to oxidize hypoxanthine to uric acid and hydrogen peroxide, wherein the methyltransferase activity is rate-limiting with respect to the nucleosidase, deamination and oxidation activities. In particular embodiments, the adenosine nucleosidase activity is rate-limiting with respect to the deamination and oxidation activities.

It should be understood that the detailed description and the specific examples included herein, are given by way of illustration only, and various changes and modifications within the spirit and scope of the invention will become apparent to those skilled in the art from the enabling teachings and description provided above and the example enzymes and assays provided herein.

### Example 1

### Methods

Expression and purification of MBP-adenine deaminase. The DNA encoding Bacillus subtilis adenine deaminase was PCR-amplified from the pHH1010 plasmid (Nygaard et al., J Bacteria 178:846-53, 1996) and ligated into a pMAL-c2x plasmid vector (New England BioLabs) between EcoR1 and SalI sites. Escherichia coli TB-1 cells were transformed with the resulting plasmid and grown aerobically in 1 L LB broth at 37° C. for 11 hours. Expression of maltose binding protein (MBP)/adenine deaminase fusion protein was induced with 0.8 mM IPTG for 10 hours. Cells were harvested by centrifugation and re-suspended in ˜30 mLs column buffer (20 mM Tris-HCl pH 7.4, 200 mM NaCl, 1 mM EDTA, and 0.2 mM DTT). Cells were lysed via sonication using four 2 min discontinuous cycles on ice using, and the cell debris and unbroken cells removed by centrifugation at 55,000×g, 4° C. for 25 minutes. The supernatant was filtered through a 0.45 μm filter and incubated with 10 mL amylose resin slurry (New England BioLabs) at 4° C. for 90 min with gentle agitation. After washing the resin with 30 mL column buffer, MBP-adenine deaminase was eluted in 5 mL fractions with column buffer containing 10 mM maltose. Fractions demonstrating >95% purity by SDS-PAGE were concentrated by Centricon-Plus Concentrators (30,000 MWCO, Amicon) and the buffer exchanged to column buffer as per the manufacturer's instructions. The purified protein was stored at −80° C. in 25% glycerol. Protein concentration was determined spectrophotometrically using ε₂₈₀ₙₘ=85,770 M⁻¹cm⁻¹. Approximately 18 mg of purified protein was obtained from 1 L of broth.

Purification of His-PRMT1. The DNA encoding rat PRMT1 was PCR amplified from the GST-PRMT1 vector with Pfu polymerase and ligated into a pET28b vector (Novagen) to yield an N-terminal His-tagged PRMT1 construct. E. coli BL21(DE3) cells carrying the pET28b/PRMT1 plasmid were grown in LB broth at 37° C. Protein expression was induced with 1.2 mM IPTG for 5 h. Cells were harvested by centrifugation and resuspended in wash buffer (50 mM sodium phosphate pH 7.5 and 20 mM imidazole). Cells were lysed by sonication using three 15 s cycles and centrifuged at 100,000×g at 4° C. for 1 h. The resulting crude supernatant was incubated with Ni Sepharose™ High Performance resin (Amersham Biosciences) for 4 h at 4° C. The slurry was loaded into a 1.7×13 cm column, and the flow through was collected. The column was washed with 65 mL wash buffer, and the protein was eluted with 10 mL of wash buffer containing 250 mM imidazole. The eluate was concentrated in Centricon-Plus Concentrators (30,000 MWCO, Amicon), the buffer exchanged to 50 mM sodium phosphate buffer pH 7.5, and 10% glycerol was added to the pure protein before storing it at −80° C. Approximately 5.4 mg pure protein was obtained from 1 L of broth and was >95% pure by SDS-PAGE.

Procedure for the enzyme-coupled photometric assay. Assays were performed in thermostatted 1 cm quartz cuvettes at 37° C. Manganese sulfate (MnSO₄) was added to a final concentration between 10 to 1050 μM. Between 10 to 1,050 μM of manganese, the same activity was observed. Manganese or other divalent ions (e.g., zinc) are required for the deaminase activity (Matsui et al., Biosci. Biotechnol. Biochem. 65:1112-1118, 2001; and Dorgan & Zhou, unpublished results). The assay involving the conversion of adenine to hypoxanthine was run using adenine at various concentrations, 1,050 μM MnSO₄, and 0.02 μM adenine deaminase buffered in 200 mM Tris pH 8.0. The assay monitoring the conversion of AdoHcy to hypoxanthine contained 54.3 μM AdoHcy, 1,050 μM MnSO₄, 0.02 μM adenine deaminase, and 17.3 nM AdoHcy nucleosidase buffered in 200 mM Tris pH 8.0. Between 3.0 to 20.0 nM adenine deaminase, the same rate was observed. Measurement of PRMT1 activity was performed in 50 mM sodium phosphate pH 7.0 with 168 μM AdoHcy nucleosidase, 0.02 μM adenine deaminase, 10-1050 μM MnSO₄, and various concentrations of PRMT1. Use of AdoHcy nucleosidase at concentrations ranging from 10 nM-168 μM yielded the same methyltransferase rate. Reactions were initiated with differing amounts of peptide substrates as indicated in the figures.

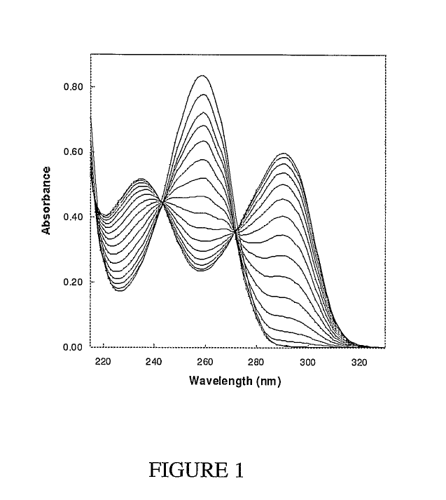

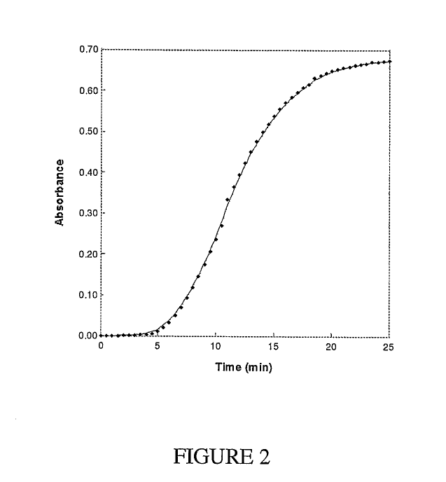

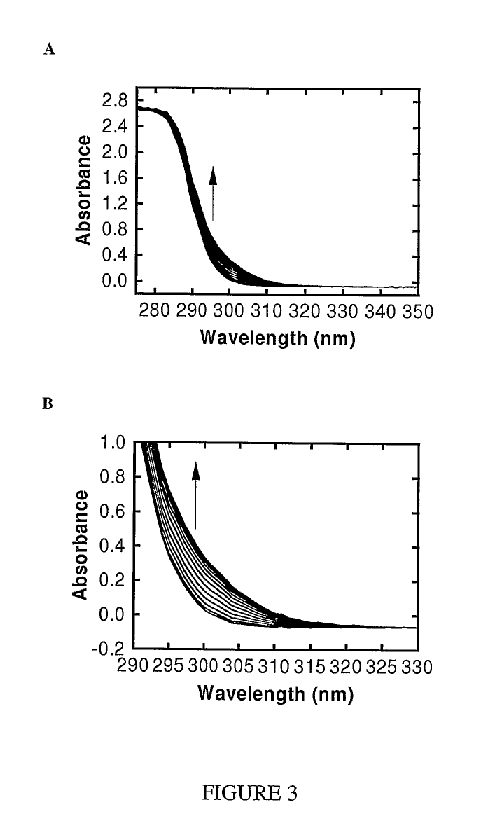

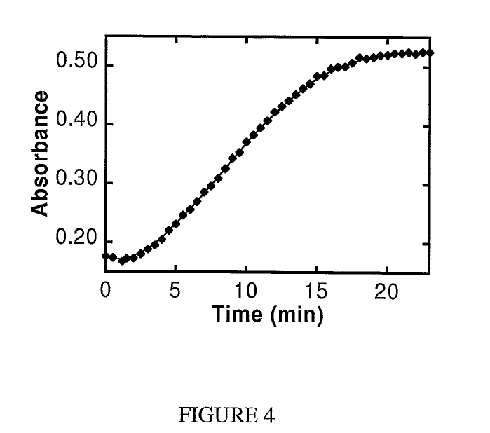

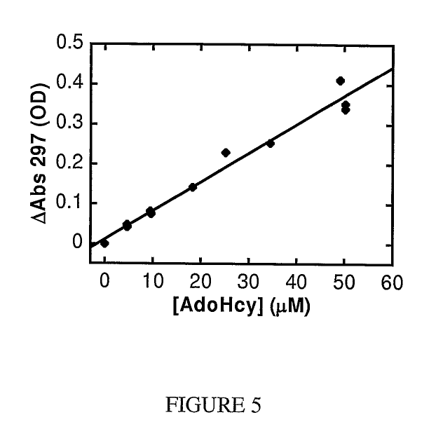

Use of Xanthine Oxidase. The reaction monitoring the conversion of AdoHcy to uric acid was performed using 54.8 μM AdoHcy, 4.1 nM xanthine oxidase, 110 μM MnSO₄, 3.0 nM adenine deaminase, and 17.3 nM AdoHcy nucleosidase buffered in 50 mM potassium phosphate pH 7.4. The AdoHcy nucleosidase reaction was rate-limiting. Reactions were initiated with AdoHcy nucleosidase. The increase in absorbance was monitored at 297 nm. As long as hydrogen peroxide (H₂O₂) produced does not affect enzyme activity, use of this oxidase in the assay pushes the readout to a longer wavelength around 297 nm, as shown in FIGS. 1 and 2. In the case of enzyme inactivation by hydrogen peroxide, commercial catalase (EC 1.11.1.6) can be added to convert hydrogen peroxide to water and oxygen, alleviating such inactivation (FIGS. 1 and 2). The assay was also investigated in the presence of dNTP in order to simulate the presence of nucleic material, such as DNA or RNA methyltransferases. The RNA and DNA substrates absorb around 260 nm and the methyltransferases absorbs around 280 nm, causing high absorbance around 280 nm at high concentrations. Thus, the utility of the xanthine oxidase step in this assay was demonstrated in the presence of up to 4 mM dNTP (data not shown). FIGS. 3 and 4 show the absorbance change of 50.2 μM AdoHcy in the presence of 1 mM each of dATP, dCTP, dGTP, and dTTP. We found that under these conditions, we could detect the conversion of AdoHcy as little as 5 μM or at even lower concentrations, as shown in FIG. 5.

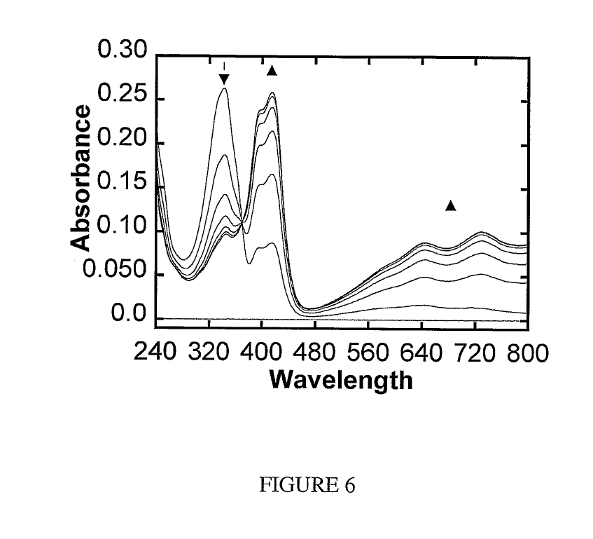

Use of Peroxidase. Peroxidase was purchased from Worthington Biochemical Corporation (Lakewood, N.J.). Assays were performed in thermostated cuvettes at 37° C. The step of the assay monitoring the conversion of 2,2′-azino-bis(3-ethylbenzothiazoline-6-sulfonic acid) (ABTS) to the ABTS radical was performed using 5.2 μM ABTS, 186 μM H₂O₂, and 0.173 nM peroxidase buffered in 50 mM potassium phosphate pH 7.4 (see FIG. 6). Next, the generation of the ABTS radical in the presence of H₂O₂ generated by xanthine oxidase was monitored using 82.1 μM hypoxanthine, 10.1 μM ABTS, 1.7 nM peroxidase, and 4.1 nM xanthine oxidase in 50 mM potassium phosphate pH 6.5. This generated a plot. Finally, the entire assay system from AdoHcy to the ABTS radical was monitored using 17.8 μM AdoHcy, 27.9 μM ABTS, 1.7 nM peroxidase, 4.1 nM xanthine oxidase, 100 μM MnSO₄, 3.0 nM adenine deaminase, and 17.3 nM AdoHcy nucleosidase buffered in 50 mM potassium phosphate pH 6.5 (Scheme 1). With this assay, applicants observed the decrease in absorbance at 260 nm and the increase in absorbance at 413 nm and higher wavelengths (indicating the formation of the ABTS radical), however the conversion was not stoichiometric and a decomposition of the product was observed after a short time.

Radioactive assay used to determine PRMT1 activity. Methyltransferase assays were equilibrated at 37° C. for 15 minutes before initiating with 211 μM R3 peptide. Each 65 μL reaction contained S-adenosyl-L-[methyl-³H] methionine (specific activity 79 μCi/μmol, Amersham Biosciences), 4 μM PRMT1, 250 μM AdoMet, 100 μM MnSO₄, 0.02 μM adenine deaminase, 168 μM AdoHcy nucleosidase, and 100 mM sodium phosphate pH 7.0. Aliquots of 10 μL were spotted directly onto P81 paper (Whatman) under vacuum in a S&S Minifold® I Slot Blot System (Schleisher & Shuell) at specific time points and washed three times with 500 μL 50 mM sodium phosphate buffer pH 7.5. After the filter paper dried, each piece was placed in 5 mL Scintisafe™ cocktail (Fisher) and counted (Beckman LS 6500). Internal tritium standards were used initially to examine counting efficiency; however, applicants determined that the presence of protein had a profound effect on counting efficiency. Instead, various volumes of the control reaction, which did not contain peptide substrate, were spotted onto P81 membranes and counted. Linearity between observed cpms and volume spotted was demonstrated. Counting efficiency was 4-6% and all calculations were adjusted accordingly. Data were plotted against time and fitted using linear regression

High performance liquid chromatograph (HPLC) analysis of reaction products. HPLC analysis of the hypoxanthine product formed during peptide methylation by PRMT1 was performed on an Apollo C-18 reverse-phase column (4.6 mm×25 cm, Alltech, Deerfield, Ill.). The column was eluted with an isocratic mixture from 0-3 minutes of 100 mM ammonium bicarbonate (A) at pH 7.8 (90%) and methanol (B) (10%). A gradient mixture was used from 3-8 minutes where the composition changed from 90% A and 10% B to 10% A and 90% B. From 8-10 minutes a gradient mixture was used and changed the composition from 10% A and 90% B to 90% A and 10% B. Finally, an isocratic mixture was used from 10-15 minutes. A 1.0 mL/min flow rate was used and the analytes (AdoMet, AdoHcy, adenine, and hypoxanthine) were monitored at 245 nm.

### Example 2

### AdoMet was Converted to Hypoxanthine Using the Coupling Enzymes, and the Coupled Enzymes Used were Shown not to be Rate Limiting

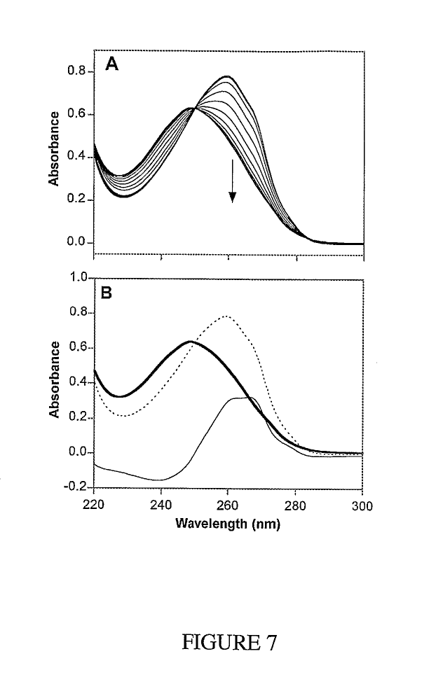

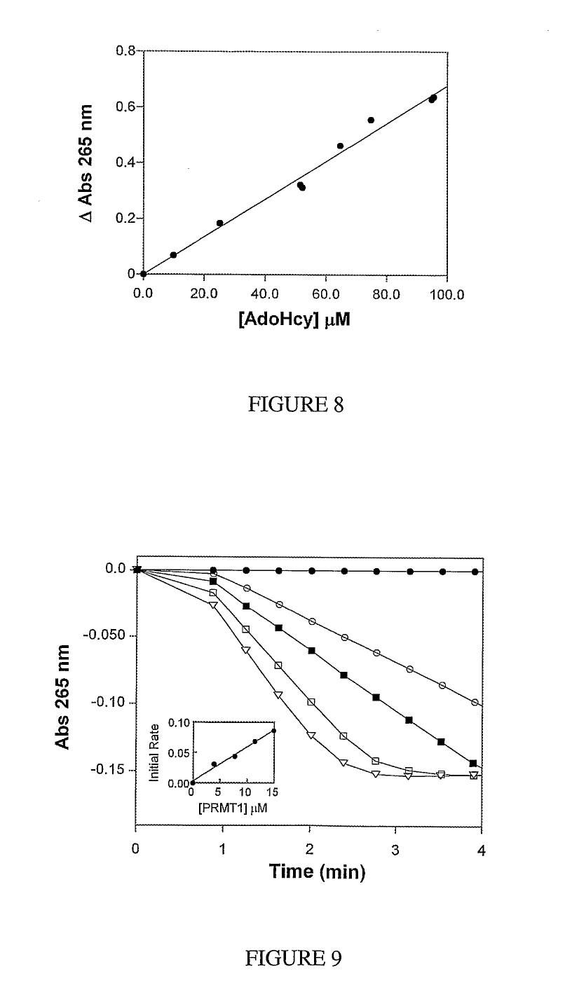

In order to yield valid kinetic parameters in the coupled assay, the coupled enzymes used should not be rate limiting, so that the measured rate is determined solely by the methyltransferase activity. The kinetics for the conversion of adenine to hypoxanthine via adenine deaminase were investigated first. Adenine absorbs maximally at 260 nm with an extinction coefficient of 13,400 M⁻¹ cm⁻¹ (The Merck Index: An encyclopedia of chemicals, drugs, and biologicals, 13^{th} Edition, M. J. O'Neil, A. Smith, P. E. Heckelman, S. Budavari (Eds.) Merck & Co., Inc., New Jersey, 2001). Upon adding adenine deaminase, an absorbance decrease at 265 nm was observed as adenine was converted to hypoxanthine rapidly in a stoichiometric fashion (FIG. 7). The k_{cat} for adenine deaminase in 100 mM Tris pH 8.0 was 35.2±0.92 sec⁻¹. The difference spectrum shown in FIG. 7B shows the maximal change in absorbance at 265 nm. In the presence of 13.3 μM adenine, concentrations of hypoxanthine ranging from 3.5 μM to 142 μM did not inhibit adenine deaminase. Complete conversion of AdoHcy to hypoxanthine using both the coupling enzymes, AdoHcy nucleosidase and adenine deaminase, was accompanied by a similar absorbance change. The reaction, like that of adenine deaminase was found to be very rapid. The k_{cat} for AdoHcy nucleosidase in 100 mM Tris, pH 8.0 was 4.12±0.10 sec⁻¹. In comparison, most AdoMet-dependent methyltransferases display k_{cat} in the 1 min⁻¹ range. The two coupling enzymes, therefore, are shown herein to be over one hundred-fold more active than most of the methyltransferases, and thus, according to particular aspects, are well suited for kinetic analysis described in this paper. The relationship between AdoHcy concentration and absorbance change at 265 nm was linear and yielded a Δε₂₆₅ of 6,700±150 M⁻¹cm⁻¹ based on the ε₂₆₀ of 15,400 M⁻¹ cm⁻¹ for AdoHcy [22] (FIG. 8).

### Example 3

### Investigation of PRMT1 Activity was Investigated Sing Exemplary Inventive Enzyme Coupled Assays

The inventive coupled methyltransferase assays were applied to the protein arginine N-methyltransferase 1 (PRMT1) as a test enzyme and a peptide corresponding to a 19-amino acid stretch of the in vivo PRMT1 protein substrate fibrillarin (Lin et al., J. Biol. Chem. 271:15034-15044, 1996). Initiation of the reaction with R3 peptide resulted in a decrease in absorbance at 265 nm as in the coupling enzyme control reactions.

FIG. 9 demonstrates that the reaction rate was dependent upon methyltransferase concentrations. The rate obtained with 2 μM PRMT1 using this continuous spectrophotometric assay with 211 μM R3 (5.1±0.2 μM AdoHyc formed/min) was verified by following [³H] incorporation from S-adenosyl-L-[methyl-³H] methionine into the R3 peptide. The rate observed using the radioactive assay was 4.9±0.6 μM AdoMet consumed/min. Furthermore, the overall reaction rates were independent on the coupling enzyme concentrations under the assay conditions. For instance, using a different preparation of PRMT1, the rates of PRMT1 catalyzed methylation of 200 μM R3 peptide using 10 nM, 100 nM, and 1 μM, 168 μM AdoHcy nucleosidase were 8.71±0.58, 9.05±0.10, 9.15±0.16 and 8.90±0.09 μM AdoMet consumed/min, respectively.

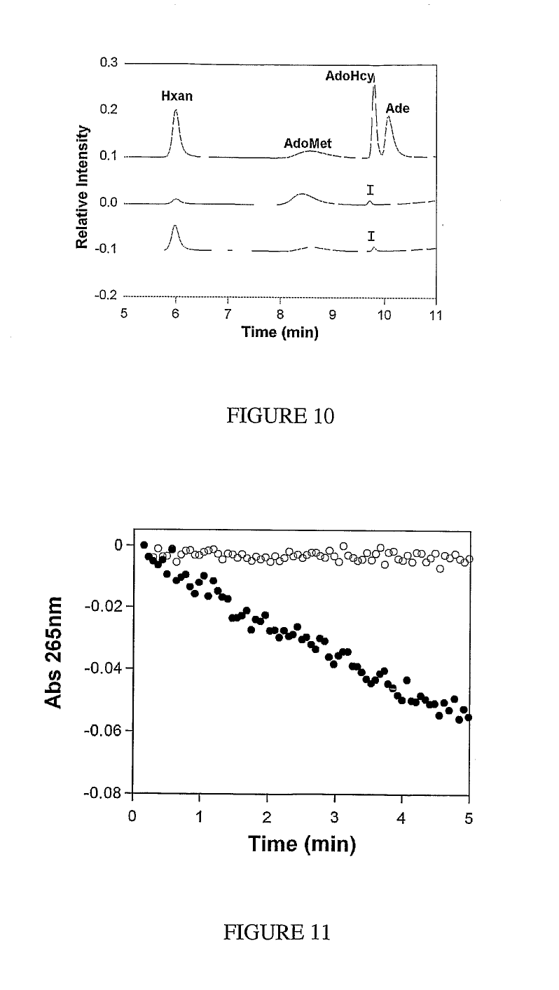

Formation of hypoxanthine and the lack of intermediate build-up during methylation of R3 by PRMT1 were confirmed using HPLC. Standards of 80 μM hypoxanthine, AdoMet, AdoHcy, and adenine were used for comparison, and were found to elute at 6.0, 8.5, 9.7, and 10.1 minutes, respectively. The formation of hypoxanthine with a corresponding disappearance of AdoMet was observed in the assay, with no detectable accumulation of the adenine or AdoHcy intermediates (FIG. 10).

Using this assay, the kinetic parameters of PRMT1 were investigated with R3 peptide from fibrillarin, an R3 analog peptide containing only one substrate arginine residue (JMH1), and H4 peptide from histone 4 (TABLE 1). The R3 peptide contains three possible arginine methylation sites, each capable of being mono and/or dimethylated (6 potential methylation events). The JMH1 peptide lacks the additional 2 substrate arginine residues of R3 but maintains the positive charge at these positions. Although Vₘₐₓ for R3 and JMH1 were similar, the Kₘ for R3 could only be estimated to be under 10 μM with this assay. A few peptide substrates have previously been used to study native PRMT1 activity and have yielded values for Kₘ of 0.2-60 μM (Hyun et al., Biochem. J. 348:573-578, 2000; and Najbauer et al., J. Biol. Chem. 268:10501-10509, 1993). The dramatic increase in V/K for R3 is most likely a result of processive methylation at multiple arginine residues on the same peptide substrate. Compared to the JMH1 peptide, the H4 peptide also contains only one arginine residue and demonstrated a K_{m,app} of 745±70 μM and Vₘₐₓ of 5.7±0.2 μM min⁻¹. These results are consistent with reports that substrates containing the RGG repeats, such as hnRNPA1 and fibrillarin, are better (catalytic efficiency) PRMT1 substrates than histone 4 (Rajpurohit et al., J. Biol. Chem. 269:1075-1082, 1994).

These data demonstrate that the inventive continuous assays can be used to characterize PRMT1 enzyme activity and, according to additional embodiments, are applicable to the assay of other AdoMet-dependent-methyltransferases. One assay described by Coward et al for catechol-O-methyltransferase (Coward and Wu, Anal. Biochem. 55:406-410, 1973) uses adenosine deaminase from Aspergillus oryzae to convert AdoHcy to S-inosylhomocysteine, however, the fungal adenosine deaminase has not been cloned, so purification of the enzyme from Taka-Diastase must be achieved with a multi-step/multi-column procedure (Sharpless and Wolfenden, Methods Enzymol. 12A:126-131, 1967). The instant exemplary assays utilizes recombinant fusion-tagged coupling enzymes that are easily purified in large amounts in a single day. More importantly, the structures of S-inosylhomocysteine and S-adenosylhomocysteine differ by only one atom (an oxygen vs a nitrogen), thus are very similar to each other. Hence, the use of MTA nucleosidase and adenine deaminase avoids any product inhibition by S-inosylhomocysteine (Coward & Slisz, J Med. Chem. 16:460-463, 1973), the product of the fungal adenosine deaminase reaction.

The inventive assays as disclosed herein are very versatile. Nonetheless, certain parameters should be noted to obtain maximal utility. For instance, if the methyltransferase substrates strongly absorb around 265 nm, a narrow range of absorbance changes will be available for activity measurement, and analysis will be subject to the detection limits of the spectrophotometer. However, even with 1 mM each of dATP, dCTP, dGTP, dTTP applicants were still able to detect the change of 35 μM of AdoHcy to hypoxanthine. Use of protein substrates exhibiting a strong UV absorbance may not be feasible. However, applicants were able to monitor methyltransferase activity using the small in vivo PRMT1 protein substrate histone 4 (FIG. 11). Several methyltransferases such as catechol O-methyltransferase display Kₘs for the methyl acceptor of ˜100 μM (Coward & Wu, Anal. Biochem. 55:406-410, 1973; Coward et al., Biochemistry 12:2291-2297, 1973), but some enzymes display much lower Kₘ values. Determination of Kₘ values lower than 10 μM may be limited by the small absorption changes at low substrate concentrations, but may be performed using progress curve analyses (Duggleby, R. G., Methods 24:168-174, 2001). In any case, the assay can be used to determine maximum rate. Finally, since AdoMet contributes to the background absorbance, concentrations of AdoMet should preferably be kept at or below 250 μM or smaller pathlength cuvettes should be used to keep the total absorption around 265 nm within the linear range of the spectrophotometer for accurate measurement.

### Example 4

### The Inventive Methods were Applied to Different AdoMet Dependent Methyltransferases (PIMT) Acting on Two Different Peptide Substrates (KASA-isoD-LAKY Peptide & (B-Asp⁵)-Delta Sleep Inducing Peptide (B-DSIP)

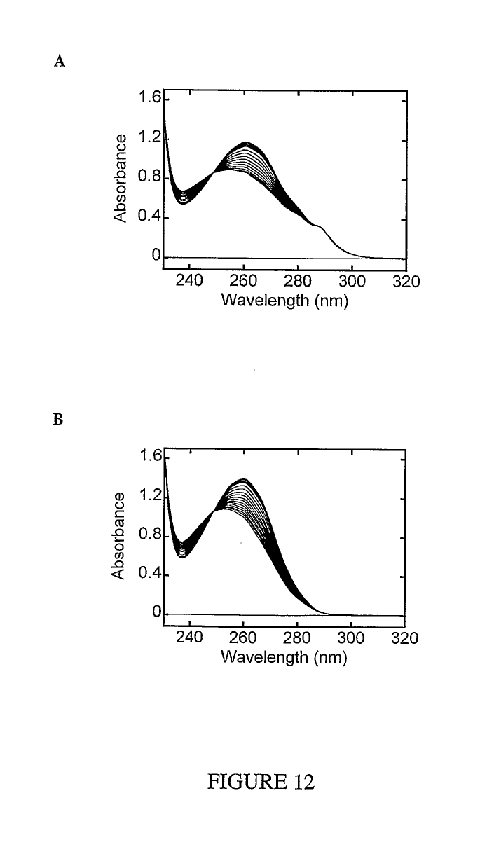

The additional exemplary embodiments the activity of Protein Isoaspartate Methyltransferase (PIMT) was monitored with two different peptides as substrates. (B-Asp⁵)-Delta Sleep Inducing Peptide (B-DSIP) was tested using 85 nM adenine deaminase, 94 μM MnSO₄, 53 nM S-adenosylhomocysteine (AdoHcy) nucleosidase, 65 μM S-adenosylmethionine (AdoMet), and 65 μM B-DSIP in 94 mM potassium phosphate, pH 7.0 at 37° C. The reaction was initiated with 110 nM PIMT and monitored at 265 nm (see FIG. 12A).

KASA-isoD-LAKY peptide was tested using 85 nM adenine deaminase, 94 μM MnSO₄, 53 nM AdoHcy nucleosidase, 94 μM AdoMet, and 51 μM KASA-isoD-LAKY peptide in 94 mM potassium phosphate, pH 7.0 at 37° C. The reaction was initiated with 110 nM PIMT and monitored at 265 nm (see FIG. 12B).

The present inventive assays have utility over a broad scope. For example, the potential for this assay goes further than AdoMet-dependent-methlytransferases. The AdoHcy/MTA nucleosidase displays broad substrate specificity cleaving not only AdoHcy and MTA, but also structural analogs with hydrophobic residues at the C5 position (Cornell et al., Biochem. Biophys. Res. Commun. 228:724-732, 1996; Lee et al., Biochemistry 43:5159-5169, 2004). In addition, the adenine deaminase also shows broad substrate specificity for purine analogs (Sakai and Jun, Pseudomonas synxantha, J. Ferment. Technol. 56:257-265, 1978; Jun and Sakai, n Pseudomonas synxantha, J. Ferment. Technol. 57:294-299, 1979). Therefore, according to further aspects, the assay is applicable to a number of other enzymes whose products can be cleaved by AdoHcy/MTA nucleosidase to generate adenine or adenine analogs that can be used by adenine deaminase. Two examples include polyamine synthesis and acylhomoserine lactone synthesis, both of which produce MTA (Fuqua et al., Annu. Rev. Genet. 35:439-468, 2001; Tabor and Tabor, Annu. Rev. Biochem. 53:749-790, 1984; incorporated herein by reference). Other enzymes can be found in a recent review on AdoMet utilizing this enzyme (Fontcave et al., Trends Biochem. Sci. 29:243-249, 2004; incorporated herein by reference).

### LITERATURE CITED

[1] H. L. Schubert, R. M. Blumenthal, X. Cheng, Many paths to methyltransfer: a chronicle of convergence, Trends Biochem. Sci. 28 (2003) 329-335.
[2] X. Cheng, R. M. Blumenthal (Eds.), S-adenosylmethionine-dependent methyltransferases: structures and functions, World Scientific Publishing Company, Singapore, 1999.
[3] X. Cheng, R. J. Robert, AdoMet-dependent methylation, DNA methyltransferases and base flipping, Nucleic Acids Res. 29 (2001) 3784-3795.
[4] M. Fujioka, Mammalian small molecule methyltransferases: their structural and functional features, Int. J. Biochem. 24 (1992) 1917-1924.
[5] Y. Zhang, D. Reinberg, Transcription regulation by histone methylation: interplay between different covalent modifications of the core histone tails, Genes Dev. 15 (2001) 2343-2360.
[6] O. F. Sarmento, L. C. Digilio, Y. Wang, J. Perlin, J. D. Harr, C. D. Allis, S. A. Coonrod, Dynamic alterations of specific histone modifications during early murine development, J. Cell Sci. 117 (2004) 4449-4459.
[7] G. L. Cuthbert, S. Daujat, A. W. Snowden, H. Erdjument-Bromage, T. Hagiwara, M. Yamada, R. Schneider, P. D. Gregory, P. Tempst, A. J. Bannister, T. Kouzarides, Histone deimination antagonizes arginine methylation, Cell 118 (2004) 545-553.
[8] U. M. Bauer, S. Daujat, S. J. Nielsen, K. Nightingale, T. Kouzarides, Methylation at arginine 17 of histone H3 is linked to gene activation, EMBO Rep. 3 (2002) 39-44.
[9] Y. Wang, J. Wysocka, J. Sayegh, Y. H. Lee, J. R. Perlin, L. Leonelli, L. S. Sonbuchner, C. H. McDonald, R. G. Cook, Y. Dou, R. G. Roeder, S. Clarke, M. R. Stallcup, C. D. Allis, S. A. Coonrod, Human PAD4 regulates histone arginine methylation levels via demethylimination, Science 306 (2004) 279-83.
[10] D. Patnaik, H. G. Chin, P. Estéve, J. Benner, S. Jacobsen, S. Pradhan, Substrate Specificity and Kinetic Mechanism of Mammalian G9a Histone H3 Methyltransferase, J. Biol. Chem. 279 (2004) 53248-53258.
[11] C. R. Creveling, J. R. Daly, Assay of enzymes of catecholamine biosynthesis and metabolism, Methods Biochem. Anal. (1971) 153-182.
[12] A. Frankel, N. Yadav, J. Lee, T. L. Branscombe, S. Clarke, M. T. Bedford, The novel human protein arginine N-methyltransferase PRMT6 is a nuclear enzyme displaying unique substrate specificity, J. Biol. Chem. 277 (2002) 3537-3543.
[13] S. Clarke, K. Banfield, S-Adenosylmethionine-dependent methyltransferase in: R. Carmel, D. W. Jacobsen (Eds.) Homocysteine in Health and Disease, Cambridge University Press, New York, 2001, pp. 63-78.
[14] M. M. Ames, C. D. Selassie, L. C. Woodson, J. A. Van Loon, C. Hansch, R. M. Weinshilboum, Thiopurine methyltransferase: structure-activity relationships for benzoic acid inhibitors and thiophenol substrates, J. Med. Chem. 29 (1986) 354-358.
[15] C. L. Hendricks, J. R. Ross, E. Pichersky, J. P. Noel, Z. S. Zhou, An enzyme-coupled colorimetric assay for S-adenosylmethionine-dependent methyltransferases, Anal. Biochem. 326 (2004) 100-105.
[16] L. M. Cannon, F. N. Butler, W. Wan, Z. S. Zhou, A stereospecific colorimetric assay for (S,S)-adenosylmethionine quanification based on thiopurine methyltransferase-catalyzed thiol methylation, Anal. Biochem. 308 (2002) 358-363.
[17] C. Wang, S. Leffler, D. H. Thompson, C. A. Hrycyna, A general fluorescence-based coupled assay for S-adenosylmethionine-dependent methyltransferases, Biochem. Biophys. Res. Commun. 331 (2005) 351-356.
[18] M. T. Bedford, S. Richard, Arginine methylation: an emerging regulator of protein function, Mol. Cell. 18 (2005) 263-272.
[19] K. A. Cornell, W. E. Swarts, R. D. Barry, M. K. Riscoe, Characterization of recombinant Escherichia coli 5′-methylthioadenosine/S-adenosylhomocysteine nucleosidase: analysis of enzymic activity and substrate specificity, Biochem. Biophys. Res. Commun. 228 (1996) 724-732.
[20] P. Nygaard, P. Duckert, H. Saxild, Role of adenine deaminase in purine salvage and nitrogen metabolism and characterization of the ade gene in Bacillus subtilis, J. Bacteriol. 178 (1996) 846-53.
[21] H. Matsui, M. Shimaoka, H. Kawaski, Y. Takenaka, O. Kurahashi, Adenine Deaminase Activity of the yicP Gene Product of Escherichia coli, Biosci. Biotechnol. Biochem. 65 (2001) 1112-1118.
[22] The Merck Index: An encyclopedia of chemicals, drugs, and biologicals, 13^{th} Edition, M. J. O'Neil, A. Smith, P. E. Heckelman, S. Budavari (Eds.) Merck & Co., Inc., New Jersey, 2001.
[23] W. Lin, J. D. Gary, M. C. Yang, S. Clarke, H. R. Herschman, The mammalian immediate-early TIS21 Protein and the Leukemia-associated BTG1 Protein Interact with a Protein-arginine N-Methyltransferase, J. Biol. Chem. 271 (1996) 15034-15044.
[24] Y. L. Hyun, D. B. Lew, S. H. Park, C. W. Kim, W. K. Paik, S. Kim, Enzymic methylation of arginyl residues in -Gly-Arg-Gly-peptides, Biochem. J. 348 (2000) 573-578.
[25] J. Najbauer, B. A. Johnson, A. L. Young, D. W. Aswad, Peptides with sequences similar to glycine, arginine-rich motifs in proteins interacting with RNA are efficiently recognized by methyltransferase(s) modifying arginine in numerous proteins, J. Biol. Chem. 268 (1993) 10501-10509.
[26] R. Rajpurohit, S. O. Lee, J. O. Park, W. K. Paik, S. Kim, Enzymatic methylation of recombinant heterogeneous nuclear RNP protein A1. Dual substrate specificity for S-adenosylmethionine:histone-arginine N-methyltransferase, J. Biol. Chem. 269 (1994) 1075-1082.
[27] J. K. Coward, F. Y. Wu, A continuous spectrophotometric assay for catechol-O-methyltransferase, Anal. Biochem. 55 (1973) 406-410.
[28] T. K. Sharpless, R. Wolfenden, A purified non-specific adenosine deaminase from Taka-Diastase, Methods Enzymol. 12A (1967) 126-131.
[29] J. K. Coward and E. P. Slisz, Analogs of S-adenosylhomocysteine as potential inhibitors of biological transmethylation. Specificity of the S-adenosylhomocysteine binding site, J Med Chem, 16 (1973) 460-463.
[30] K. Luger, T. J. Rechsteiner, A. J. Flaus, M. M. Waye, T. J. Richmond, Characterization of nucleosome core particles containing histone proteins made in bacteria, J Mol Bio, 272 (1997) 301-311.
[31] J. K. Coward, E. P. Slixz, F. Y. Wu, Kinetic studies on catechol O-methyltransferase. Product inhibition and the nature of the catechol binding site, Biochemistry 12 (1973) 2291-2297.
[32] R. G. Duggleby, Quantitative analysis of the time courses of enzyme-catalyzed reactions, Methods 24 (2001) 168-174.
[33] J. E. Lee, E. C. Settembre, K. A. Cornell, M. K. Riscoe, J. R. Sufrin, S. E. Ealick, P. L. Howell, Structural Comparison of MTA Phosphorylase and MTA/AdoHcy Nucleosidase Explains Substrate Preferences and Identifies Regions Exploitable for Inhibitor Design, Biochemistry 43 (2004) 5159-5169.
[34] T. Sakai, H. K. Jun, Purification and Characterization of Adenine Deaminase in Pseudomonas synxantha, J. Ferment. Technol. 56 (1978) 257-265.
[35] H. K. Jun, T. Sakai, Some Properties of Adenine Deaminase in Pseudomonas synxantha, J. Ferment. Technol. 57 (1979) 294-299.
[36] C. Fuqua, M. R. Parsek, E. P. Greenberg, Cell-to-Cell Communication: Acyl-Homoserine Lactone Quorum Sensing, Annu. Rev. Genet. 35 (2001) 439-468.
[37] C. W. Tabor, H. Tabor, Polyamines, Annu. Rev. Biochem. 53 (1984) 749-790.
[38] M. Fontcave, M. Atta, E. Mulliez, S-Adenosylmethionine: Nothing Goes to Waste, Trends Biochem. Sci. 29 (2004) 243-249.

1. A quantitative method for assaying methyltransferase activity, comprising:
including, in a reaction mixture having a methyl donor substrate and a methyltransferase activity that catalyses conversion of the methyl donor substrate to a transmethylation product that comprises an adensosine or adenosine derivative moiety, a purified or recombinant adenosine nucleosidase activity that catalyses release of the respective adenine or adenine derivative moiety from the transmethylation product, and a purified or recombinant adenine deaminase activity that catalyses deamination of the released moiety to hypoxanthine or respective derivative thereof and ammonia, wherein the methyltransferase activity is rate-limiting with respect to the coupled nucleosidase and deamination reactions; and
determining the methyltransferase activity by spectrophotometric or chromatographic monitoring of the coupled deamination reaction products, or of subsequent enzymatic or chemical reactions coupled thereto.

2. The method of claim 1, wherein the adenosine nucleosidase activity is rate-limiting with respect to the coupled deamination reaction.

3. The method of claim 1, wherein spectrophotometric monitoring comprises spectrophotometric monitoring of the deamination reaction.

4. The method of claim 3, wherein spectrophotometric monitoring comprises continuous monitoring of absorbance at 265 nanometers, and wherein the progress of deamination is accompanied by decreasing absorbance at 265 nanometers.

5. The method of claim 1, wherein the methyltransferase activity comprises S-adenosyl-L-methionine (AdoMet/SAM)-dependent methyltransferase activity.

6. The method of claim 1, wherein the transmethylation product comprises at least one selected from the group consisting of S-adenosylhomocysteine (AdoHcy); 5′-methylthioadenosine (MTA); and structural analogs of AdoHcy or MTA with hydrophobic residues at the C5 position.

7. The method of claim 1, where the purified or recombinant adenosine nucleosidase activity comprises a purified or recombinant S-adenosylhomocysteine (AdoHcy) nucleosidase activity.

8. The method of claim 7, wherein the purified or recombinant S-adenosylhomocysteine (AdoHcy) nucleosidase activity comprises AdoHcy nucleosidase EC 3.2.2.9.

9. The method of claim 1, wherein the purified or recombinant adenine deaminase activity comprises adenine deaminase EC 3.5.4.2.

10. The method of claim 1, further comprising coupled oxidation of the hypoxanthine to uric acid and hydrogen peroxide using purified or recombinant xanthine oxidase, wherein the methyltransferase activity is rate-limiting with respect to the coupled nucleosidase, deamination and oxidation reactions, and wherein determining the methyltransferase activity comprises spectrophotometric or chromatographic monitoring of the coupled oxidation reaction.

11. The method of claim 10, wherein the adenosine nucleosidase activity is rate-limiting with respect to the coupled deamination and oxidation reactions.

12. The method of claim 10, wherein spectrophotometric monitoring of the coupled oxidation reaction comprises continuous monitoring of absorbance at 295 or 297 nanometers, and wherein the progress of oxidation reaction is accompanied by increasing absorbance at 295 or 297 nanometers.

13. The method of claim 10, wherein the purified or recombinant xanthine oxidase is included in the reaction mixture.

14. The method of claim 10, wherein the purified or recombinant xanthine oxidase comprises xanthine oxidase EC 1.1.3.22.

15. The method of claim 10, further comprising conversion of the hydrogen peroxide to water and oxygen using purified or recombinant catalase.

16. The method of claim 10, further comprising peroxidation of the hydrogen peroxide using purified or recombinant peroxidase, and wherein determining the methyltransferase activity comprises spectrophotometric or chromatographic monitoring of the peroxidation reaction.

17. The method of claim 16, wherein spectrophotometric monitoring of the peroxidation reaction comprises conversion of 2,2′-azino-bis(3-ethylbenzothiazoline-6-sulfonic acid) (ABTS) to the ABTS radical and monitoring of an increase in absorbance at 413 nm or at a higher wavelength characteristic of the formation of the ABTS radical.

18. The method of claim 1, wherein the adenine deaminase activity is a recombinant adenine deaminase activity.

19. The method of claim 18, wherein the recombinant adenine deaminase activity is a recombinant fusion-tagged adenine deaminase.

20. A kit for assaying of methyltransferase activity; comprising:
a purified or recombinant adenosine nucleosidase activity suitable to catalyse release of an adenine or adenine derivative moiety from a transmethylation product of a transmethylation reaction; and
a purified or recombinant adenine deaminase activity suitable to catalyse deamination of the released moiety to hypoxanthine or respective derivative thereof and ammonia, wherein the methyltransferase activity is rate-limiting with respect to the nucleosidase and deamination activities.

21. The kit of claim 20, wherein the adenosine nucleosidase activity is rate-limiting with respect to the deamination activity.

22. The kit of claim 20, further comprising a purified or recombinant xanthine oxidase suitable to oxidize hypoxanthine to uric acid and hydrogen peroxide, wherein the methyltransferase activity is rate-limiting with respect to the nucleosidase, deamination and oxidation activities.

23. The kit of claim 22, wherein the adenosine nucleosidase activity is rate-limiting with respect to the deamination and oxidation activities.
